SEQUENCE LISTING (16 sequences)

SEQ ID NO: 1 - DNA, 22 bp, artificial
  synthetic primer
accgagaatatgaatcatggtg

SEQ ID NO: 2 - DNA, 22 bp, artificial
  synthetic primer
ttctcgccatccgaatatatcc

SEQ ID NO: 3 - DNA, 23 bp, artificial
  synthetic primer
tggacccgtgaactacccacagc

SEQ ID NO: 4 - DNA, 20 bp, artificial
  synthetic primer
atatcctgcttggtcactcc

SEQ ID NO: 5 - DNA, 24 bp, artificial
  synthetic primer
tcgccctgaccaccatgctcagcc

SEQ ID NO: 6 - DNA, 20 bp, artificial
  synthetic primer
tcggggatcctccagatggt

SEQ ID NO: 7 - DNA, 21 bp, artificial
  synthetic primer
tcccctccagcggtcaatgtc

SEQ ID NO: 8 - DNA, 23 bp, artificial
  synthetic primer
acccgagaacacacaggacattc

SEQ ID NO: 9 - DNA, 18 bp, artificial
  synthetic primer
accattgtgccctgcttc

SEQ ID NO: 10 - DNA, 24 bp, artificial
  synthetic primer
atcactcctctctttacatcatgc

SEQ ID NO: 11 - DNA, 18 bp, artificial
  synthetic primer
tagcggcagggagactac

SEQ ID NO: 12 - DNA, 21 bp, artificial
  synthetic primer
ggtcagcatcactgttaagga

SEQ ID NO: 13 - DNA, 22 bp, artificial
  synthetic primer
ttctacaatgagctgcgtgtgg

SEQ ID NO: 14 - DNA, 19 bp, artificial
  synthetic primer
aagctgtagccacgctcgg

SEQ ID NO: 15 - DNA, 18 bp, artificial
  synthetic primer
caccagtggaagtccaca

SEQ ID NO: 16 - DNA, 20 bp, artificial
  synthetic primer
ttcccgttgctgtactcccc

(19) United States
(12) Patent Application Publication
(10) Pub. No.: US 2014/0024038 A1
(43) Pub. Date: Jan. 23, 2014

(54) METHOD AND REAGENTS FOR TREATING HEPATIC FIBROSIS AND INFLAMMATION

(71) Applicant: Agency for Science, Technology and Research, Singapore (SG)

(72) Inventors: Gunter Maubach, Singapore (SG); Lang Zhuo, Singapore (SG); Michelle Chin Chia Lim, Singapore (SG)

(21) Appl. No.: 13/949,143

(22) Filed: Jul. 23, 2013

Related U.S. Application Data
(63) Continuation of application No. 12/279,984, filed on Aug. 2, 2010.
(60) Provisional application No. 60/774,543, filed on Feb. 21, 2006.

(51) Int. Cl. C12Q 1/37

(52) U.S. Cl. CPC C12Q 1/37
USPC 435/6.12; 435/7.4; 435/23

(57) ABSTRACT

The invention relates to methods for identifying an anti-fibrotic or anti-inflammatory agent comprising determining cathepsin S expression in activated hepatic stellate cells which have been exposed to a test compound and comparing expression to non-exposed hepatic stellate cells. The invention also relates to methods for treating a disorder characterised or caused by hepatic fibrosis or inflammation, comprising administering a cathepsin S inhibitor to a subject.

### CROSS REFERENCE TO RELATED APPLICATIONS

This application claims the benefit of U.S. provisional patent application No. 60/774,543, filed Feb. 21, 2006, the contents of which are herein fully incorporated by reference.

### FIELD OF THE INVENTION

This invention relates generally to methods and reagents for treating hepatic fibrosis and inflammation.

### BACKGROUND OF THE INVENTION

Hepatic stellate cells (HSCs), representing 5-8% of total liver cells, are found in the space of Disse of adult livers between hepatocytes and liver sinusoidal endothelial cells. The classical functions of HSCs are fat storage, vitamin A uptake and metabolism. The basic pathobiology and history of HSC discovery have been reviewed elsewhere (Burt, 1999; Sato et al. 2003). During the past decades, many authors have shown that HSCs play an important role in defending liver from injuries and at the same time are mediators of hepatic fibrosis by producing profibrotic cytokines and extracellular matrix (ECM) proteins (Sato et al., 2003; Bataller & Brenner, 2001; Lotersztajn et al., 2005). These different functions of HSCs are tightly linked to their transition from a quiescent to an activated phenotype.

Activation of HSCs is a dominant event in fibrogenesis. During activation, quiescent vitamin A storing cells are converted into proliferative, fibrogenic, proinflammatory and contractile ‘myofibroblasts’ (Friedman, 2003; Bataller & Brenner, 2001; Cassiman et al., 2002). HSC activation proceeds along a continuum that involves progressive changes in cellular function. Early events in activation render the cells responsive to cytokines and other local stimuli. The earliest change in stellate cells reflects the paracrine stimulation by all neighboring cell types (Friedman, 2003). Activated HSCs show de novo fibrogenic properties, including proliferation and accumulation in areas of parenchymal cell necrosis, secretion of proinflammatory cytokines and chemokines, and synthesis of a large panel of matrix proteins and of inhibitors of matrix degradation, leading to progressive scar formation (Lotersztajn et al., 2005). In vivo, activated HSCs migrate and accumulate at the sites of tissue repair, secreting large amounts of ECM components and regulating ECM degradation (Cassiman et al., 2002). HSCs are believed to play a role in the pathogenesis of a number of clinically important conditions such as, for example, hepatic fibrosis, cirrhosis, portal hypertension and liver cancer (Geerts, 2004). Hence, HSCs have also become a target for the development of anti-fibrotic therapies (Bataller & Brenner, 2001; Bataller & Brenner, 2005; Friedman, 2003)

### SUMMARY OF THE INVENTION

In one aspect there is provided a method of identifying an anti-fibrotic agent, the method comprising: (a) determining a first expression level of Cat S in a first activated hepatic stellate cell; (b) exposing a second activated hepatic stellate cell to a test compound; (c) determining a second expression level of Cat S in said second hepatic stellate cell; (d) comparing the first expression level and the second expression level, whereby the first expression level which is greater than the second expression level indicates that the test compound is an anti-fibrotic agent.

In another aspect, there is provided a method of identifying an anti-inflammatory agent, the method comprising: (a) determining a first expression level of Cat S in a first activated hepatic stellate cell; (b) exposing a second hepatic stellate cell to a test compound; (c) determining a second expression level of Cat S in said second activated hepatic stellate cell; (d) comparing the first expression level and the second expression level whereby the first expression level greater than the second expression level indicates that the test compound is an anti-inflammatory agent.

In another aspect, there is provided a method for treating a disorder characterized or caused by hepatic fibrosis or inflammation in a subject, the method comprising administering to the subject a cathepsin S inhibitor.

In another aspect, there is provided use of a cathepsin S inhibitor for the treatment of a disorder in a subject, the disorder characterized or caused by hepatic fibrosis or inflammation.

In another aspect, there is provided use of a cathepsin S inhibitor in the preparation of a medicament for the treatment of a disorder characterized or caused by hepatic fibrosis or inflammation.

In another aspect, there is provided a kit comprising, (a) a hepatic stellate cell; and (b) a reagent for detecting the expression level of Cat S.

### BRIEF DESCRIPTION OF THE DRAWINGS

In the figures, which illustrate by way of example only, embodiments of the present invention:

FIG. 1 depicts epifluorescece microscope images of rat HSCs stained with various HSC markers (Panels A to C) and with DAPI (Panel D);

FIG. 2 depicts ethidium bromide staining of reverse-transcribed—PCR products from HSC-T6 cells after agarose gel electrophoresis;

FIGS. 3A-C depict a quantitative analysis of the effects of IFN-γ on the identified mRNAs (Cat S, CD74, CIITA, RT1-Dα) in HSC-T6 cells (FIG. 3A), activated HSCs (FIG. 3B) and CFSC-3H cells (FIG. 3C) as determined by real-time RT-PCR;

FIG. 4 depicts a quantitative analysis of the effects of IFN-γ on cathepsin L RNA levels in HSC-T6 cells, activated HSCs and CFSC-3H cells;

FIG. 5 depicts immunofluorescence images of the effect of IFN-γ on the expression of CD74 in HSC-T6 cells and activated HSCs;

FIG. 6 depicts immunofluorescence images of the effect of IFN-γ on the expression of RT1-B in HSC-T6 cells and activated HSCs;

FIG. 7 depicts immunofluorescence images of the effect of IFN-γ on the expression of cathepsin S in HSC-T6 cells and activated HSCs;

FIG. 8 depicts a quantitative comparison of the effects of IFN-γ on the immunofluorescence intensities of the indicated proteins in HSC-T6 cells and activated HSCs;

FIG. 9 depicts the effect of IFN-γ on cathepsin S activity in HSC-T6 cells, activated HSCs and CFSC-3H cells; and

FIG. 10 depicts epifluorescence images of the uptake and processing of DQ-ovalbumin by HSC-T6 cells and activated HSCs.

### DETAILED DESCRIPTION

Antigen presentation via MHC class II is a complex process. The early stage of this process involves the induction of the class II transactivator (CIITA), which is the ‘master regulator’ of the MHC class II expression. CIITA responds to different proinflammatory stimuli and induces the expression of the classical MHC class II molecules (RT1-B, RT1-D) as well as the accessory molecule invariant chain (CD74, also known as li-chain) (for a review, see LeibundGut-Landmann et al., 2004) After induction, the assembly of class II molecules (RT1-Bα and β, RT1-Dα and β) with CD74, a type II membrane protein, occurs followed by the stepwise processing of CD74 into CLIP (class II associated li-peptide) starting from the C-terminus. The invariant chain directs the MHC class II complex to the late endocytic compartment and prevents the premature loading of the antigen-binding groove.

There are many different proteases involved in the processing of CD74. The most effective proteases involved in the last step of this process are cathepsin S and L. These enzymes release CLIP from lip10 (leupeptin induced polypeptide). Depending on the cell type, cathepsin L and cathepsin S are involved in this step-wise degradation of the invariant chain in thymic epithelial cells (Nakagawa et al., 1998) and in B-cells, macrophages and dendritic cells (Riese et al., 1998; Beers et al., 2005; Driessen et al., 1999), respectively.

Liver fibrosis is a common outcome of chronic hepatic inflammation and the role of HSCs in fibrosis is undoubted. When the liver is injured, a regulated wound healing process involving HSCs occurs which is coupled with an inflammatory response, e.g. damaged cells release certain factors, in response to these factors neighbouring cells, like Kupffer cells, start to secret cytokines which attract leucocytes, which in turn generate lipid peroxides, reactive oxygen species (ROS). Hepatic injury leads to inflammation which, in turn, activates HSCs and ECM production which, upon continued stimuli, leads to fibrosis.

Recent papers have shown the antigen presentation capability of the HSCs (Vifias et al., 2003; Yu et al., 2004; Winau et al., 2007). This finding is important as most cell types of the liver contribute to the immune response of the liver in different ways. Hepatocytes produce acute phase proteins (Gabay & Kushner, 1999; Wigmore et al., 1997) and liver sinusoidal endothelial cells induce tolerance (Limmer et al., 2000). The Kupffer cells, resident macrophages of the liver, and resident dendritic cells are known professional antigen-presenting cells (Shiratori et al., 1984; Roland et al., 1994; O'Connell et al., 2000; Johansson & Wick, 2004; Lau & Thomson, 2003). Villas et al. (2003) and Yu et al. (2004) describe the presence of surface molecules (HLA-DR) and co-molecules (CD40, CD80, CD86), which are necessary for antigen presentation to the T-cells, on the HSCs. In particular, these surface molecules and co-molecules are upregulated when HSCs are treated with IFN-γ. It was further demonstrated that HSCs were capable of inducing T-cell proliferation, although less efficiently when compared to the professional antigen presenting cells (APCs), such as Kupffer or dendritic cells, of the liver. No information, however, was available concerning the early events of antigen presentation in HSCs or about the molecules involved in these events.

The inventors have surprisingly discovered that activated HSCs express products necessary for the early stage of antigen presentation, including cathepsin S. Cathepsin S expression is upregulated by interferon γ (IFN-γ). Activated HSCs, as well as the HSC cell lines HSC-T6 and CFSC-3H expressed transcripts for all molecules studied, namely CIITA, RT1-Bα, RT1-Dα, CD74 and cathepsin S. Further, we discovered that semi-activated and in vivo activated HSCs were capable of taking up antigenic proteins and possess the molecular machinery to process them into smaller peptides. The finding that Cat S is expressed and active in HSC's places the HSC's not only in the role of wound healing, but in the processes of inflammation, and fibrosis. We have therefore determined that selective inhibition of cathepsin S activity in HSCs can provide a mechanism for modulating hepatic immunity, and thus inflammation and fibrosis.

There is thus presently provided a method of identifying an anti-fibrotic agent, the method comprising comparing the Cat S expression levels in activated HSCs in the presence and absence of a test compound.

There is also provided a method of identifying an anti-inflammatory agent, the method comprising comparing the Cat S expression levels in activated HSCs in the presence and absence of a test compound.

In brief, the method entails determining the Cat S expression level in activated HCSs not exposed to a test compound and determining the Cat S expression level exposed to a test compound and comparing the expression levels. The expression level in the activated HSCs not exposed to the test compound, if greater, is indicative of the test compound being an anti-fibrotic or anti-inflammatory agent. The expression levels can be determined in a known manner as further described below. The agents identified by the methods described herein can be used as hepatic anti-fibrotic and anti-inflammatory agents and as anti-fibrotic and anti-inflammatory agent in other cells and organs in which similar stellate cell type as HSC is present, for example, in cells of the pancreas, kidney, brain known or expected to also express cathepsin S.

Cathepsin S is a lysosomal cysteine endoprotease involved in the proteolytic processing of lip10 to CLIP in certain APCs. In vitro, cathepsin S can mediate all of the digestion steps of class II-li complexes. Cathepsin S is highly expressed in professional APCs, such as, for example B cells and dendritic cells. Cathepsin S activity is essential for the maturation of dendritic cells required for the strong stimulation of T-lymphocytes (Driessen et al., 1999).

As used herein, “HSC” includes a primary hepatic stellate cell (or cells) isolated from liver, as well as cells derived from the in vitro passage of primary HSCs. Methods for isolating primary HSCs would be known to a person skilled in the art, for example, those described in Friedman et al. (1992) and Cassiman et al., (1999). Unless the context dictates otherwise, as used herein “HSC” includes both quiescent and activated HSCs. Activated HSCs may be obtained by known methods, such as, for example, by culturing primary HSCs on uncoated plastic substrates.

Primary HSCs may be isolated from liver by known methods. As used herein, “HSC” also includes model HSC-derived cell or cells, such as, for example, the immortalized rat HSC-T6 cell. Rat HSC-T6 cells exhibit an activated phenotype reflected in their fibroblast-like shape, rapid proliferation in culture and the expression of desmin, smooth muscle alpha action (SMAA), glial fibriallery acidic protein (GFAP) and vimentin (Vogel et al., 2000). Other HSC-derived model cell lines would be known to a person skilled in the art and include, for example, the human LX-1, LX-2 cell lines (Xu et al., 2005) and CFSC-3. Both LX-1 and LX-2 cell lines express a number of markers of activated HSC, including SMAA and GFAP. HSC-T6, LX-1 and LX-2 cells may be deactivated by growth in Matrigel™ or by culture in low serum media (Xu et al., 2005). The CFSC-3 line is derived from a CCl₄ induced cirrhotic liver in Wistar male albino rats and is considered and in vivo activated HSC line. In a specific embodiment, the HSC is HSC-T6.

A test compound will be exposed to an activated hepatic stellate cell typically by incubating the stellate cell with the test compound for a period of time necessary to observe the effect of the test compound on the Cat S expression, if any. The test compound may be exposed to the activated stellate cell in any other manner that permits any such effect to be determined. In certain embodiments the test compound, which may be a solid, a liquid, a suspension or a solution, is added or admixed to a culture comprising the second stellate cell. The length of time the second stellate cell is exposed to the test compound may depend on a number of factors, and may be on the order of minutes, hours or days. A person skilled in the art would know or readily determine how long to expose a second stellate cell to a test compound, for instance by determining the effect of the test compound as a function of time. In various embodiments, the stellate cell is exposed to the test compound between 2 and 48 hours prior to the determination of the Cat S mRNA and protein expression level in the stellate cell.

A skilled person would readily be able to determine the appropriate concentration of a test compound, for example with reference to IC₅₀ of compounds known to reduce the expression level of Cat S, (the concentration required to effect 50% reduction in the expression). The skilled person will also appreciate that a compound with a lower IC₅₀ is a more potent inhibitor of Cat S expressions. As would be known to a person skilled in the art, the concentration of the test compound used in the method should be sufficient to observe detectable reduction in Cat S expression so as to avoid a false negative result attributed to insufficient concentration. In various embodiments, the amount of the test compound exposed to the second stellate cell results in a concentration of the test compound in the picomolar (10⁻¹² M) to nanomolar (10⁻⁹ M) range.

In certain embodiments, the HSC is provided. In specific embodiments, the HSC is provided in vitro. In some embodiments, the HSC provided in vitro is a HSC-T6 cell.

In some embodiments, the HSC cell is exposed to a cytokine prior to determining the expression levels in the absence and presence of a test compound. As will be understood cytokine generally refers to water-soluble proteins and glycoproteins with a mass of generally of approximately 8-30 kDa. Cytokines may be autocrine, paracrine or endocrine. Appropriate cytokines would be known to a person skilled in the art and include, for example, interferon-γ (IFN-γ), TNF-α, epidermal growth factor (EGF), TGF-β, IL-1. In a specific embodiment, the cytokine is IFN-γ.

As used herein, “expression” refers to any detectable level in the Cat S transcription or translation product in a HSC. As will be understood by a person skilled in the art, transcription levels may be determined by direct methods that measure the amount of Cat S mRNA, for example, Northern Blotting or quantitative RT-PCR. Alternatively, Cat S expression may be determined indirectly by measuring the optical, coulorometric, fluorogenic, enzymatic or immunogenic properties of the cathepsin S protein. In various embodiments, Cat S expression is determined by Western blotting/analysis or immunofluorescence techniques employing an anti-Cat S antibody. The anti-cathepsin S antibody may be monoclonal or polyclonal. Polyclonal anti-cathepsin S antibodies may be obtained from commercial sources (BioVision). A person of skilled in the art would readily know how to determine the expression level of Cat S, either on a mRNA or on a polypeptide level.

The test compound may be any reagent that may inhibit cathepsin S activity. Test compounds may be designed de novo by methods known to a person skilled in the art. For example, the crystal structure of cathepsin S has been determined and test compounds may be selected based on modeling calculations suggesting that the test compound may potently and or selectively bind to the active site of cathepsin S (see, for example, Katunuma et al., 1999). Such modeling programs are commercially available, and would be known to a person skilled the art.

Alternatively, the compound may be identified by screening libraries of compounds. Such libraries may be created by known combinatorial chemistry approaches or may be obtained commercially. As would be known to a person skilled in the art, small molecule test compounds are generally preferred over larger compounds.

There is also provided a method for treating a disorder characterized or caused by hepatic fibrosis or inflammation in a subject, the method comprising administering to the subject a cathepsin S inhibitor. Use of a cathepsin S inhibitor in such treatment and in the manufacture of a medicament for such treatment is also contemplated.

The term “treating” or “treatment” of a disorder characterized or caused by hepatic fibrosis or inflammation refers to an approach for obtaining beneficial or desired results, including clinical results. Beneficial or desired clinical results can include, but are not limited to, alleviation or amelioration of one or more symptoms or conditions, diminishment of extent of disorder, stabilization of the state of disorder, prevention of development of disorder, delay or slowing of disorder progression, delay or slowing of disorder onset, amelioration or palliation of the disorder, and remission (whether partial or total). “Treating” can also mean prolonging survival of a patient beyond that expected in the absence of treatment.

“Treating” can also mean inhibiting the progression of the disorder, slowing the progression of the disorder temporarily, although more preferably, it involves halting the progression of the disorder permanently.

Disorders caused or characterized by hepatic fibrosis or inflammation would be known to a person skilled in the art and include, for example, cirrhosis, portal hypertension, live cancer, hepatitis C infection, hepatitis B infection, autoimmune hepatitis steatohepatitis associated with alcohol or obesity, hemochromatosis, Wilson's disorder, primary biliary cirrhosis (PBC) and non-alcoholic steatohepatitis (NASH), hepatic rejection, including auto-immune rejection and rejection after organ transplant, and chronic live rejection.

As used herein, a “cathepsin S inhibitor” contemplates a molecule or molecules that decrease the proteolytic activity of cathepsin S. Cathepsin S inhibitors may act directly by decreasing or inhibiting enzymatic turnover. Without being limited to any particular mode of action, cathepsin inhibitors may form irreversible covalent enzyme-inhibitor complexes with cathepsin S. In some embodiments, the cathepsin S inhibitor is morpholinurea-leucine-homophenylalanine-vinylsulfone-phenyl (LHVS) (Riese et al., 1998), trans-epoxysuccinyl-1-leucylamido-(4-guanidino) butane (E-64) or CLIK [II] 60 (Katunuma et al., 1999). The cathepsin S inhibitor may also be a non-covalent inhibitor, such as, for example, 1-[3-[4-(6-Chloro-2,3-dihydro-3-methyl-2-oxo-1H-benzimidazol-1-yl)-1-piperidinyl]propyl]-4,5,6,7-tetrahydro-5-(methylsulfonyl)-3-[4-(trifluoromethyl)phenyl]-1H-pyrazolo[4,3-c]pyridine (JNJ 10329670) (Thurmond et al., 2004).

As used herein, a cathepsin S inhibitor also contemplates reagents that decrease or reduce Cat S mRNA levels, including reagents that inhibit Cat S transcription, or activate Cat S mRNA degradation. Without being limited to any particular theory, examples of such cathepsin inhibitors include nucleic acid based inhibitors. In some embodiments, the cathepsin S inhibitor is a ribozymes, antisense RNAs, or micro RNAs. Peptide nucleic acid (PNA) analogues of these inhibitors are also contemplated.

In other embodiments, the Cat S inhibitor is a siRNA. siRNAs are generally double stranded 19 to 22 nucleotide sequences that can effect post-transcriptional silencing of cognate mRNAs, allowing for selective suppression of gene expression. Generally, and without being limited to any specific theory, the sequence of the siRNA therapeutic product will be complementary to a portion of the mRNA of the gene sought to be silenced. For example, the siRNA may be designed to hybridize with a mRNA encoding Cat S.

Guidelines for designing siRNAs would be known to the person skilled in the art, or siRNA designed to hybridize to a specific target may be obtained commercially (Ambion, Qiagen). For example, siRNAs with a 3′ UU dinucleotide overhang are often more effective in inducing RNA interference (RNAi). Other considerations in designing siRNAs would be known to a person skilled in the art.

Nucleic acid-based cathepsin S inhibitor may be made by known methods, for examples by chemical synthesis or may be obtained from commercial sources.

The subject of the method may be any subject in need of treatment. In some embodiments, the subject is a human subject.

The cathepsin S inhibitor is administered in an effective amount to achieve the desired treatment, for example, to inhibit HSC cathepsin S activity. For example, cathepsin S inhibitor may be delivered in such amounts to inhibit, partially or completely, cathepsin S, which functions to alleviate, mitigate, ameliorate, inhibit, stabilize, improve, prevent, including slow the progression of the disorder, the frequency of treatment and the type of concurrent treatment, if any.

To aid in administration, a cathepsin S inhibitor may be formulated as an ingredient in a pharmaceutical composition. The compositions may routinely contain pharmaceutically acceptable concentrations of salt, buffering agents, preservatives and various compatible carriers or diluents. The cathepsin S inhibitor may be formulated in a physiological salt solution.

The proportion and identity of the pharmaceutically acceptable diluent is determined by chosen route of administration, compatibility with a nucleic acid molecule, compatibility with a live virus when appropriate, and standard pharmaceutical practice. Generally, the pharmaceutical composition will be formulated with components that will not significantly impair the biological properties of cathepsin S inhibitor. Suitable vehicles and diluents are described, for example, in Remington's Pharmaceutical Sciences (Remington's Pharmaceutical Sciences, Mack Publishing Company, Easton, Pa., USA 1985).

Solutions of a cathepsin S inhibitor may be prepared in a physiologically suitable buffer. Under ordinary conditions of storage and use, these preparations may contain a preservative to prevent the growth of microorganisms, but that will not inactivate or degrade the cathepsin S inhibitor. A person skilled in the art would know how to prepare suitable formulations. Conventional procedures and ingredients for the selection and preparation of suitable formulations are described, for example, in Remington's Pharmaceutical Sciences and in The United States Pharmacopeia: The National Formulary (USP 24 NF19) published in 1999.

The forms of the pharmaceutical composition suitable for injectable use include sterile aqueous solutions or dispersion and sterile powders for the extemporaneous preparation of sterile injectable solutions or dispersions, wherein the term sterile does not extend to any live virus that may comprise the nucleic acid molecule that is to be administered. In all cases the form must be sterile and must be fluid to the extent that easy syringability exists.

Kits and commercial packages (useful, for example, for identifying an antifibrotic or anti-inflammatory agent, including hepatic anti-fibrotic and anti-inflammatory agent) comprising activated HSCs, for example, HSC-T6 and a reagent for detecting Cat S expression are also contemplated. In various embodiments, the reagent for detecting Cat S expression may be a nucleic acid complimentary to all or a portion of the Cat S mRNA. As would be understood by a person skilled in the art the complimentary polynucleotide should be long enough to allow for selective hybridization to the Cat S mRNA. In other embodiments, the reagent for detecting Cat S expression is an anti-cathepsin antibody which may be monoclonal. The nucleic acids and antibodies may be labeled by methods known in the art to assist in their detection. Such a kit or commercial package may also contain instructions regarding use of the activated HSC and the reagent for detecting Cat S expression and for identifying an anti-fibrotic or anti-inflammatory agent. Such kits may also contain a cytokine such as IFN-γ.

As can be understood by one skilled in the art, many modifications to the exemplary embodiments described herein are possible. Such modifications include the substitution of known equivalents for any aspect of the invention to achieve substantially the same result in substantially the same way. The invention, rather, is intended to encompass all such modification within its scope, as defined by the claims.

All references and documents referred to herein are fully incorporated by reference.

### EXAMPLES

### Materials and Methods

Isolation of Primary HSCs

The primary HSCs were isolated from Wistar rats according to a previously published protocol (L. Riccalton-Banks et al., 2003). Briefly, the supernatant was centrifuged at 50×g for 5 min for several rounds until no visible pellet was observed. The next centrifugation step at 200×g for 10 min yielded a pellet containing the HSCs. This pellet was washed once in culture medium and recovered by centrifugation.

The cells were re-suspended in culture medium and seeded into 75 cm² culture flasks. For immunocytochemistry, some cells were plated onto glass cover slips in a 24-well culture plate.

Cell Culture

The rat HSC cell line HSC-T6 (Vogel et al., 2000) was a gift from Dr. Scott Friedman Mount Sinai School of Medicine in New York). The cell line CFSC-3H was kindly provided by Dr. Marcus Rojkind at Albert Einstein College of Medicine, Bronx, N.Y. The HSC cell lines and the primary rat HSCs were routinely cultured in DMEM (Dulbecco's Modified Eagle Medium), supplemented with 10% FBS and 100 U penicillin/100 μg/ml streptomycin at 37° C. in a humidified atmosphere of 5% CO2. The HSC cell lines were split twice a week in a 1:3 ratio by trypsinization (0.05% trypsin/0.53 mM EDTA). The primary cells were passaged when required. The primary cells used in all experiments are cell culture activated. All the cell culture media and reagents were purchased from Invitrogen (CA, USA).

IFN-γ Treatment and RNA Isolation

HSC-T6, CFSC-3H and activated HSCs were plated into 75 cm² culture flasks and grown overnight at conditions described above. The cells at a confluence of 60-70% were incubated with 10 ng/ml final concentration (equivalent to 100 U/ml) of recombinant rat IFN-γ for 2, 4, 8 and 24 h. The recombinant rat interferon-γ (IFN-γ) was purchased from BioVision (CA, USA). Total RNA was isolated using the NucleoSpin RNAII isolation kit (Macherey & Nagel, Germany) according to the manufacturer's protocol. The RNA concentration was measured with the ND-100 spectrophotometer (NanoDrop Technologies, DE, USA).

OneStep RT-PCR

RT-PCR was performed with the OneStep RT-PCR kit from Qiagen (Germany). 100 ng to 1 μg of total RNA was used in each RT-PCR reaction, depending on the abundance of the transcript. Table 1 shows the sequence of the primers used. The primer concentration used was 0.6 μM, as recommended by the manufacturer. The Q-solution was included in all RT-PCR reactions to minimize nonspecific products. The RT step was carried out for 30 min at 42° C. and followed by deactivation for 15 min at 95° C. Conditions for PCR were as follow: 10 s for denaturation at 94° C., 10 s for annealing at an appropriate temperature, and 19 s for synthesis at 72° C. A total of 40 cycles were performed. We used the PTC-200 thermal cycler (MJ Research, FL, USA). The products were analyzed by electrophoresis in a 3% agarose gel and visualized with ethidium bromide staining. A 100 by DNA ladder (SM0242, Fermentas, Lithuania) was used as a size marker in all gels. DNA sequencing confirmed the identity of all PCR products. Sequencing service was performed by Research Biolabs Singapore.

Reverse Transcription and Real-Time PCR of Cat S, Cat L, CD74, CIITA and RT1-Da

Real-time PCR was performed using the ABI 7500 Real Time PCR System (Applied Biosystems, CA, USA). All reagents were purchased from this company unless otherwise stated. Total RNA was reverse transcribed to cDNA using reagents from the cDNA archive kit (4322171). 10 μg of total RNA was used in a total reverse transcription reaction volume of 100 μl. The RT step was performed for 10 min at 25° C. and 2 h at 37° C. In real-time PCR, 20× TaqMan gene expression assay mix of Cat S (Rn01534427_m1), Cat L (Rn00565793_m1), CD74 (Rn01491430_g1), CIITA (Rn01424723_g1) and RT1-Dα (Rn02346209g1), as well as 20×18s rRNA (4319413E) were used. For each real-time target, the reaction comprises of 3 μl cDNA, 0.5 μl 20×18S rRNA, 0.5 μl 20× TaqMan gene expression assay, 1 μl nuclease-free water and 5 μl TaqMan Universal PCR master mix (4352042). Conditions for PCR were 2 min 50° C., 10 min 95° C. and 40 cycles of 15 s 95° C. and 1 min 60° C. The comparative threshold method was used to quantitate relative changes of target mRNA (User Bulletin #2, Applied Biosystems). Relative quantitation of target mRNA was expressed as fold change in gene expression to control (untreated). The data presented are representative for three independent experiments with the same trend. The graphs were made using OrignPro 7 (OriginLab, MA, USA).

Immunostaining and Microscopy

The primary cells were grown in DMEM supplemented with 10% FBS on glass cover slips in 24-well culture plates prior to staining with antibodies against GFAP, SMAA, Desmin, RECA-1, ED-2, cathepsin S, RT1-B and CD74. At 60-70% confluence, the cells were washed once with sterile PBS and fixed with 4% paraformaldehyde (PFA) for 30 min at 4° C., followed by three washes with PBS. The cells were blocked and permeabilized in blocking solution (10% horse serum, 0.1% Triton X-100 in PBS) for 1 h at 37° C. The primary antibody was incubated in 10% blocking solution in PBS for 1 h at 37° C. followed by the secondary antibody under the same conditions. The primary antibody was omitted as a negative control. All images were taken with the LEICA DM IRB epifluorescence microscope using a 63× objective.

For induction experiments, IFN-γ was added to the cell culture at low confluence and the cells were cultivated further for different times depending on the targets to be stained (Cat S: 0, 4, 8, 24, 30 h; CD74: 0, 8, 24, 30 h; and RT1-B: 0, 24, 48 h). The presented images are representative for 3 experiments. Images were taken with a 40× objective. The fluorescence intensities from 3 to 7 different fields of vision of one representative experiment were quantified using the Image processing toolbox of the MATLAB platform (MathWorks, MA, USA). The following antibodies were used in the immunofluorescent staining. Anti-Cat S antibody (sc-6505) and anti-CD74 antibody (sc-5438) were from Santa Cruz Biotechnology (CA, USA); anti-RT1-B antibody (554926) was from BD Pharmingen (CA, USA); anti-GFAP antibody (Z 0334) was from Dako (CA, USA); and anti-SMAA-Cy3 (C 6198) and antidesmin antibody (D 8281) were from Sigma Chemicals (MO, USA). The anti-RECA-1 antibody (MCA970) and anti-ED-2 (CD163, MCA342R) were from Serotec (Oxford, UK). The secondary antibodies used were anti-goat-Alexa488 (A-21467, Invitrogen),

Cathepsin S Activity Measurement

The cells were grown in cell culture medium containing 10% FBS in 75 cm² flasks. When the cells reached 50-60% confluence, IFN-γ was added at 10 ng/ml or omitted as untreated sample. The cells were harvested 48 h later and resuspended in CS cell lysis buffer. Cathepsin S activity was measured using a kit from BioVision (K1101-01) according to the provided manual. The final substrate concentration was 200 μM. Emitted fluorescence was measured using the Tecan Satire II (Tecan, Zurich, Switzerland) fluorescence plate reader at λₑₓ: 400 nm and λₑₘ: 505 nm. An AFC standard curve was used to calculate the released fluorophore in μM per μg protein per hour at 37° C.

Antigen Uptake and Processing Experiment

The cells were grown to 70% confluence and incubated with 100 μg/ml DQ-ovalbumin (Invitrogen) for 15 min at 37° C. or 4° C. (control) and then washed twice with medium. The cells were further incubated at 37° C. and mounted onto microscope slides after different time points. The uptake and digest of the tracer ovalbumin was imaged with the LEICA DM IRB epifluorescence microscope using the FITC filter. Representative images of 3 independent experiments were shown.

Results

HSC Isolation and Characterization

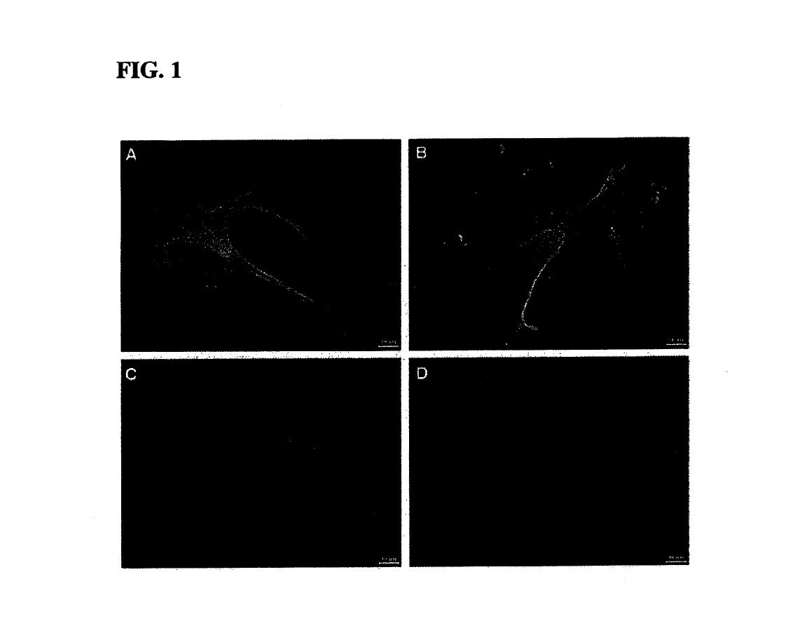

Primary HSCs were isolated from Wistar rat livers according to a previously published protocol (Riccalton-Banks et al., 2003) and seeded into 75 cm² culture flasks or onto glass cover slips. At the same time, part of the cell pellet was also used for total RNA isolation. To confirm the HSC identity, primary cells cultured for 3 days on glass cover slips were stained with antibodies against GFAP, desmin, and SMAA respectively. As illustrated in FIG. 1, cells in short-term (3 days) culture displayed prominent filamentous GFAP staining (FIG. 1A) in numerous (but not all) cells, along with staining for two other HSC markers, desmin (FIG. 1B) and SMAA (FIG. 1C). FIG. 1A-1C were counterstained with DAPI (blue). The scale bar in FIG. 1 is 10 μm. In the subsequent 7 days, the cells gradually lost the strong filamentous GFAP staining. At the same time, cells acquired very pronounced SMAA staining, with a typical filamentous distribution. Notably the intense SMAA staining even extended to the nucleus (FIG. 1D), suggesting that the HSCs are at a highly activated state. The purity of our HSC preparation was estimated to be greater than 95% according to GFAP positive staining. In addition, the cells were stained negative for RECA-1 antigen, a marker for endothelial cells. A small percentage of cells stained positive for ED-2 (CD163), a Kupffer cell marker, within the first few days. In order to confirm that the activated HSCs used in our study were not contaminated by Kupffer cells we performed a RT-PCR using primers for a specific Kupffer cell marker (77- to 88-kDa fucose receptor). The RT-PCR could not detect this marker within 30 cycles.

Transcriptional Expression of Early Molecules Required for Antigen Presentation in HSCs

In order to investigate whether HSCs express the main molecules required in the beginning of antigen presentation at the transcriptional level, specific RT-PCR primer pairs (Table 1) were designed.

TABLE 1

| Primer | | | |
|---|---|---|---|
| (bp) (° C.) | Annealing | Sequence | Length |
| sCathepsin S | 55 | 5′-ACCGAGAATATGAATCATGGTG-3′ | 127 |
| asCathepsin S | | 5′-TTCTCGCCATCCGAATATATCC-3′ | |
| asCD74. | 59 | 5′-TGGACCCGTGAACTACCCACAGC-3′ | 234 |
| asCD74 | | 5′-ATATCCTGCTTGGTCACTCC-3′ | |
| sRT1-Bα | 55 | 5′-TCGCCCTGACCACCATGCTCAGCC-3′ | 187 |
| asRT1-Bα | | 5′-TCGGGGATCCTCCAGATGGT-3′ | |
| sRT1-Dα | 55 | 5′-TCCCCTCCAGCGGTCAATGTC-3′ | 259 |
| asRT1-Dα | | 5′-ACCCGAGAACACACAGGACATTC-3′ | |
| sCIITA type I | 55 | 5′-ACCATTGTGCCCTGCTTC-3′ | 243 |
| sCIITA type III | 52.4 | 5′-ATCACTCCTCTCTTTACATCATGC-3′ | 130 |
| sCIITA type IV | 55 | 5′-TAGCGGCAGGGAGACTAC-3′ | 141 |
| asCIITA type I, III, IV | | 5′-GGTCAGCATCACTGTTAAGGA-3′ | |
| sβ-actin | 55 | 5′-TTCTACAATGAGCTGCGTGTGG-3′ | 332 |
| asβ-actin | | 5′-AAGCTGTAGCCACGCTCGG-3′ | |
| sCathepsin L | 55 | 5′-CACCAGTGGAAGTCCACA-3′ | 122 |
| asCathepsin L | | 5′-TTCCCGTTGCTGTACTCCCC-3′ | |

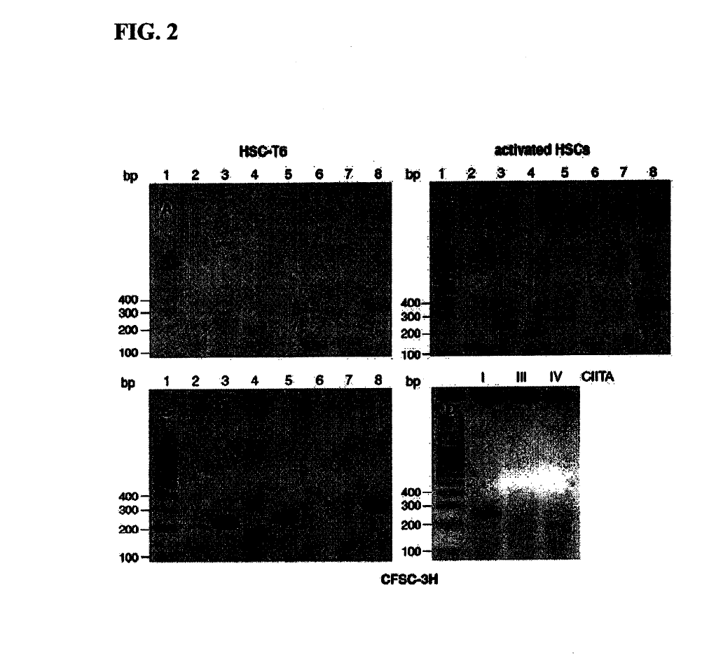

The molecules studied included the class II transactivator (CIITA), which is the major transcriptional regulator of MHC class II molecules, being a transcriptional co-activator; the MHC class II molecules (RT1-Dα and RT1-Bα) themselves; the invariant chain (CD74), a chaperone for the MHC class II molecules, and cathepsin S, which is a lysosomal protease predominantly expressed in antigen presenting cells and lymphatic tissues, and has been implicated in the processing of the invariant chain in certain cell types (Riese et al., 1998; Beers et al., 2005; Driessen et al., 1999). We included an established cell line derived from a CCl4-induced fibrotic liver in our study in order to investigate whether a different history of activation makes a difference with respect to the expression of the studied molecules. Total RNA were isolated from primary HSCs that had been culture activated for 36 days, as well as from the HSC-T6 and CFSC-3H cell lines cultured in the absence of IFN-γ. As shown in FIG. 2, the activated HSCs and both cell lines had the same expression pattern for Cat S, CD74, RT1-Bα and RT1-Dα and showed a basal transcript level of these molecules.

FIG. 2 depicts the results of oneStep RT-PCR analysis of key molecules involved in the early steps of antigen presentation. Total RNA extracted from the HSC-T6 cell line (FIG. 2A), primary HSCs cultured for 36 days (FIG. 2B) and CFSC-3H (FIG. 2C) was analyzed by RT-PCR using primers indicated in Table 1. Lane 1: 100 by MW ladder (bp), 2: cathepsin S, 3: invariant chain (CD74), 4: RT1-Bα, 5: RT1-Dα, 6: CIITA type IV, 7: CIITA type III, and 8: β-actin. All products detected had the expected size and were confirmed by sequencing. Because we did not detect the mRNA for CIITA types III and IV in CFSC-3H, a RT-PCR for CIITA type I was performed (FIG. 2D). This demonstrates that important molecules involved in the early stage of antigen presentation are expressed in HSCs.

Interestingly, both type IV CIITA, (known as the major IFN-γ inducible transactivator (Steimle et al., 1994) and type III CIITA (known to regulate the constitutive class II expression in B cells (Lennon et al., 1997)) were expressed in activated HSCs, as well as in HSC-T6 (FIGS. 2A and 2B), but not in CFSC-3H (FIG. 2C).

Because we were unable to detect the CIITA types III and IV in the CFSC-3H cell line, we tested whether they are expressing the type I. Indeed we found the sole expression of type I in this cell line (FIG. 2D). In contrast, type I was not expressed in HSC-T6 and activated HSCs. Notably the expression of cathepsin S (Flannery et al., 2003) and CIITA type III (Soos et al., 2001) was also shown for gliomas.

The mRNA for cathepsin L, another lysosomal cysteine protease, which could be involved in the CD74 processing, was present in all cells used.

Quantitative Analysis of IFN-γ Effect on mRNA Transcripts of Cat S, Cat L, CD74, CIITA and RT1-Dα

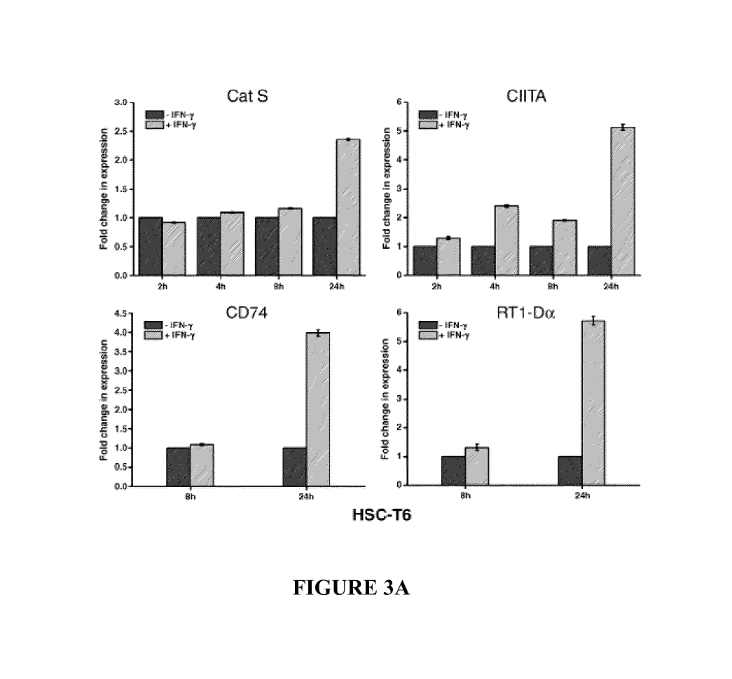

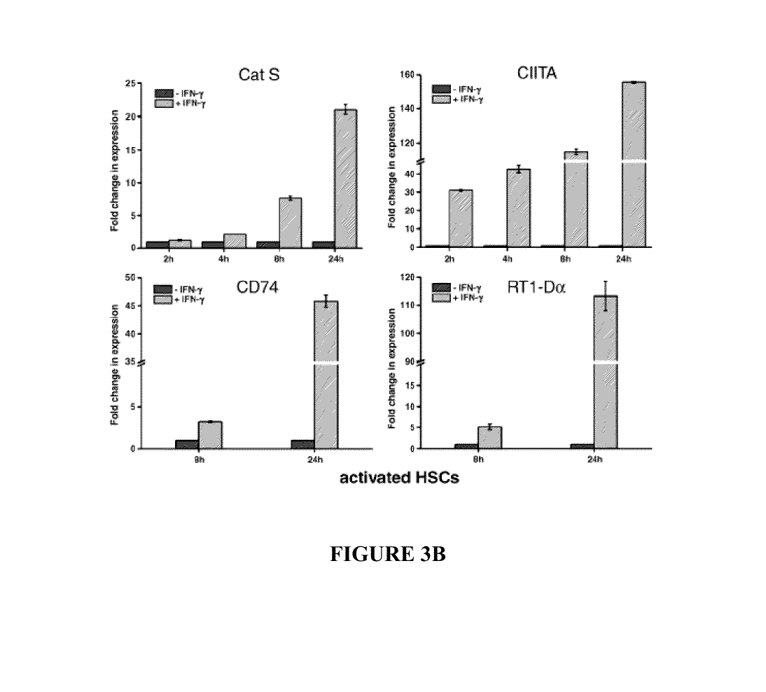

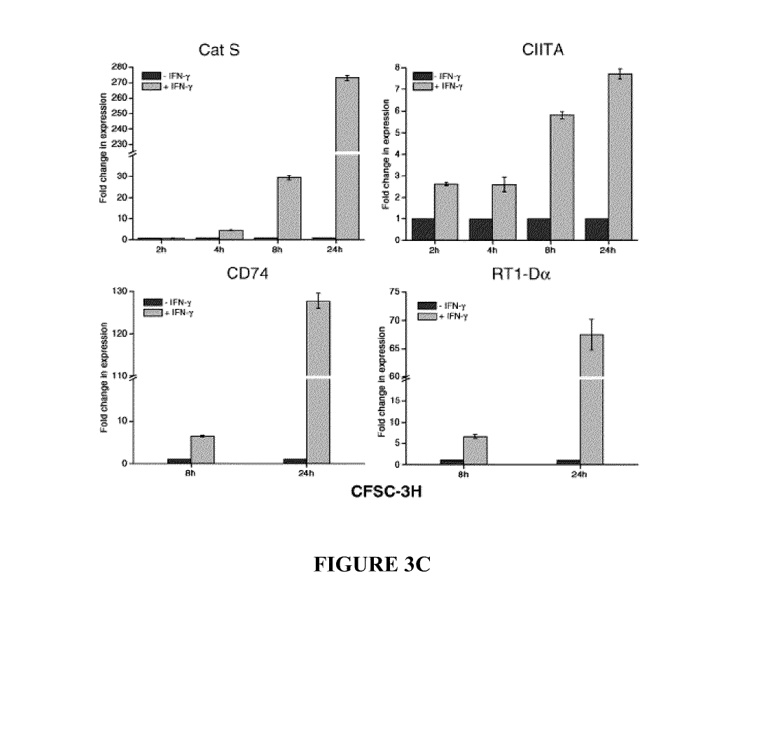

FIG. 3A-3C depicts a quantitative analysis of the change in cathepsin S, CIITA, CD74 and RT1-Dα transcript level upon treatment with IFN-γ using real-time RT-PCR. After treatment with IFN-γ for 2, 4, 8 and 24 h, total RNA was extracted from the three different HSCs (activated HSC, HSC-T6 and CFSC-3H). The RNA was reverse transcribed and real-time RT-PCR was performed using TaqMan assays. The results were expressed as fold change in gene expression compared to the untreated samples using the relative quantification method for HSC-T6 (FIG. 3A), activated HSCs (FIG. 3B) and CFSC-3H (FIG. 3C). The upregulation of all transcripts by IFN-γ is demonstrated for all studied HSCs.

The transcripts for CIITA, CD74, RT1-Dα and Cat S were detected by quantitative real-time RT-PCR using Taqman assays (FIG. 3A-C) and are presented as fold change in gene expression relative to the untreated sample.

Upon induction with IFN-γ, we observed that the CIITA (note that the Taqman assay detects all variants) and the cathepsin S transcripts in HSC-T6 (FIG. 3A), activated HSCs (FIG. 3B) and CFSC-3H (FIG. 3C) started to increase at an early time point, which was in general agreement with earlier observations (Storm van's Gravesande et al., 2002; Rahat et al., 2001). While the increase in the CIITA transcript level was similar in HSC-T6 and CFSC-3H, it was more then 20 times higher in activated HSCs. Even though the cathepsin S transcription was upregulated by IFN-γ (somewhat slower for the HSC-T6), there is an order of magnitude difference in the change of the mRNA level in the following order HSCT6<activated HSC<CFSC-3H.

As expected, the mRNA expression of the MHC class II molecule (RT1-Dα), and the invariant chain (CD74) were also induced, but indirectly through the subsequent action of CIITA at a later time. This type of activation by CIITA is reviewed in Harton et al., 2000. As can be seen from the graphs (FIG. 3A-C), the fold change in gene expression for CD74 and RT1-Dα compared to the control is much lower in HSC-T6 than in activated HSCs and CFSC-3H. The difference is about 10 to 20 times.

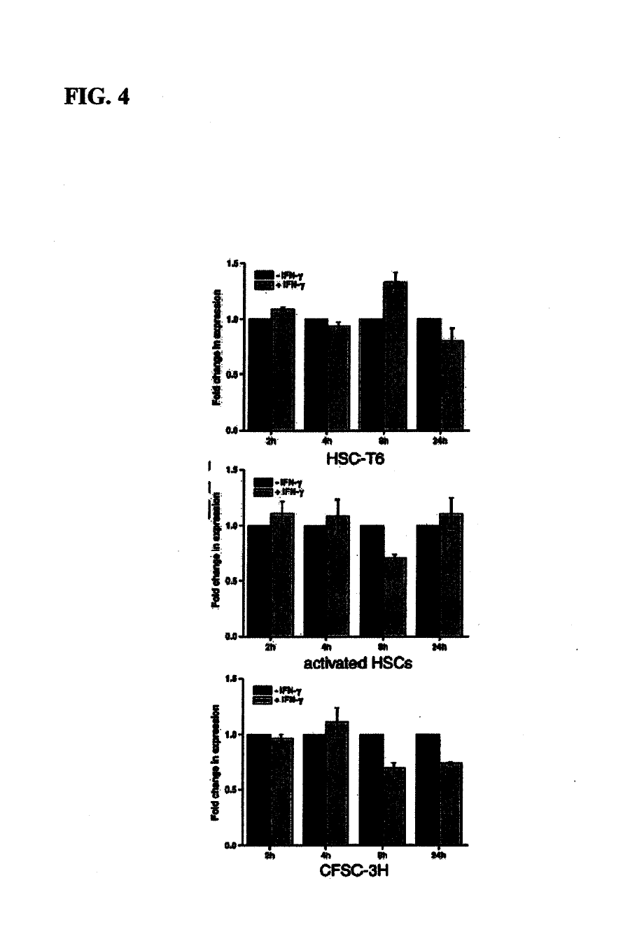

Cathepsin L is expressed in activated HSCs, HSC-T6 and CFSC-3H. Because cathepsin L is used in the thymus for the processing of CD74, we wanted to see if the cathepsin L mRNA is upregulated in hepatic stellate cells. Contrary to the observation for cathepsin S, IFN-γ treatment had no influence on cathepsin L mRNA level. In fact, after 8 h, the cathepsin L mRNA expression decreased in CFSC-3H (FIG. 4). In FIG. 4, the cells were treated with IFN-γ for 2, 4, 8 and 24 h, total RNA was extracted from activated HSCs, HSC-T6 and CFSC-3H respectively. After reverse transcription, the cathepsin L mRNA level was analyzed using real-time RT-PCR. Using the relative quantification method, the results were expressed as fold change in gene expression compared to the untreated samples. The graphs illustrate the lack of change in cathepsin L mRNA level after treatment with IFN-γ.

Upregulation of CD74, RT1-B and Cathepsin S Proteins

In order to show that the increase in mRNA level also reflects an increase in protein expression, immunofluorescence staining was used to assess the respective proteins in both the untreated and IFN-γ treated cells at different time points. For these experiments, we used the HSC-T6 and the activated HSCs. FIG. 5 depicts the effect of IFN-γ treatment on the expression of CD74 in HSC-T6 cells and activated HSCs. HSC-T6 and activated HSCs were plated and grown to a confluence of 60-70% overnight at conditions described. The cells were treated with IFN-γ for a different period of time. FIG. 5A and FIG. 5C show the immunofluorescence staining with anti-CD74 antibody after 30 h of IFN-γ induction for HSC-T6 and activated HSCs respectively (arrows depict the increase in fluorescence). FIG. 5B and FIG. 5D display the controls without IFN-γ for HSC-T6 and activated HSCs respectively. Cells were counterstained with DAPI (blue). The scale bars in the images of FIG. 4 are 10 μm.

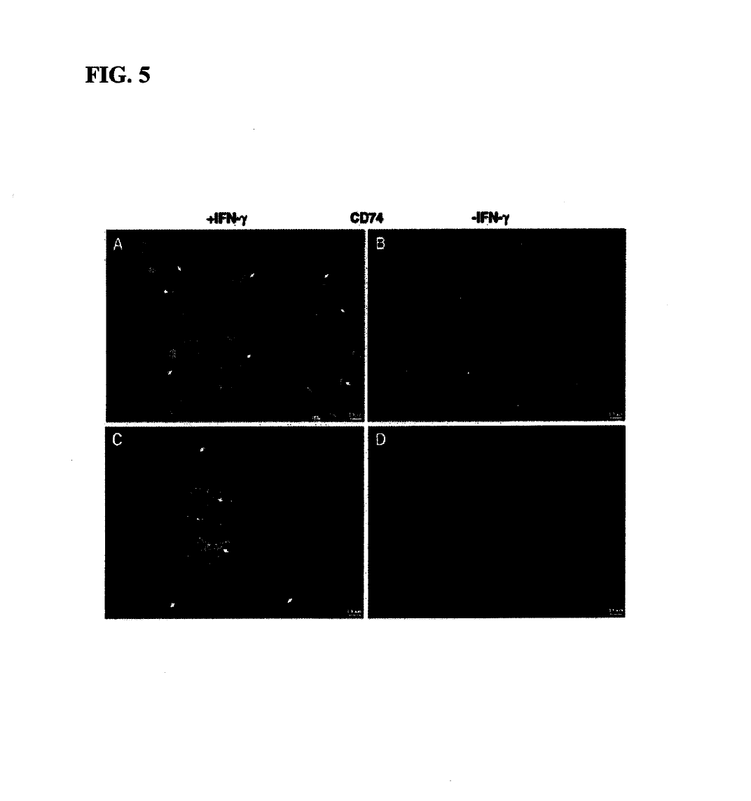

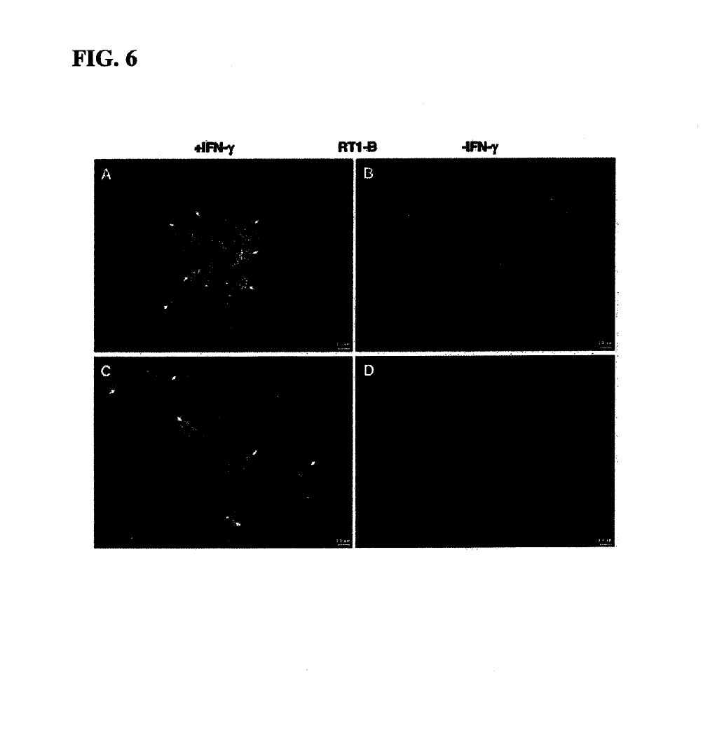

Upregulation of CD74 expression under IFN-γ treatment was observed for HSC-T6 (FIG. 5A) and activated HSCs (FIG. 5C). CD74 expression was significantly induced by IFN-γ after 30 h, as shown by arrows in the micrographs, and exhibited a typical perinuclear staining. This observation is consistent with the trafficking pattern of membrane-targeted proteins. In contrast, HSCs under untreated conditions showed a much weaker or barely detectable staining of CD74 (FIG. 5B, D). Similarly, the expression of RT1-B in both the HSC-T6 cell line (FIG. 6A) and activated HSCs (FIG. 6C) was also induced after 48 h of IFN-γ treatment. FIG. 6 depicts IFN-γ effect on the expression of MHC class II molecule RT1-B in HSC-T6 and activated HSCs. HSC-T6 and activated HSCs were plated and grown to a confluence of 60-70% overnight at conditions described. The cells were treated with IFN-γ for 48 h and immunologically stained as described. FIG. 6A and FIG. 6C show the immunofluorescence of the anti-RT1-B antibody after 48 h of induction, for HSC-T6 and activated HSCs respectively (arrows show the increase in immunofluorescence). FIG. 6B and FIG. 6D are the respective controls without addition of IFN-γ. Cells were counterstained with DAPI (blue). The scale bars in FIG. 6 are 10 μm. Newly synthesized RT1-B proteins were visible in the perinuclear region (depicted by arrows). This observation is consistent with ER/Golgi localization. Interestingly the immunofluorescence staining for CD74 and RT1-B was never homogenously distributed over the cells.

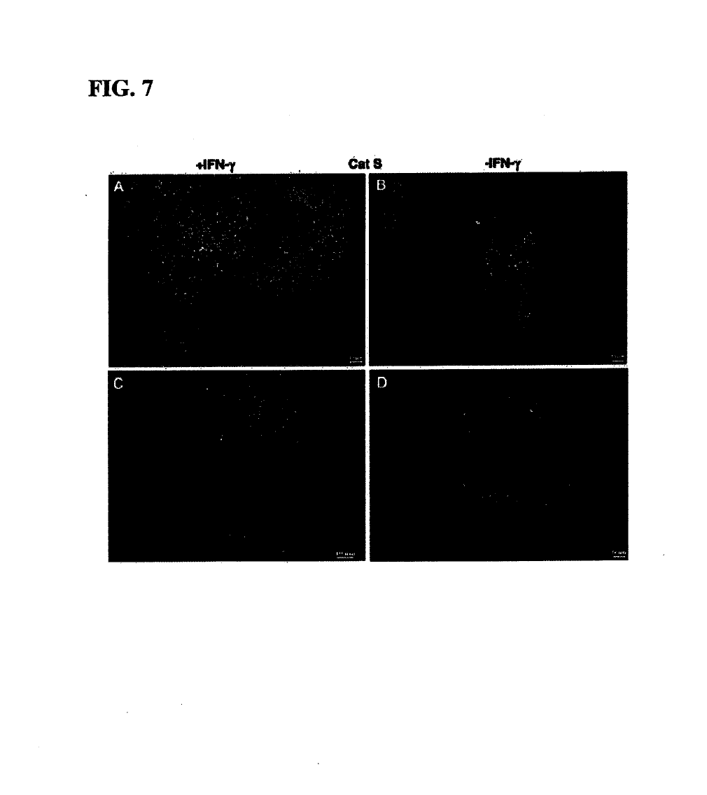

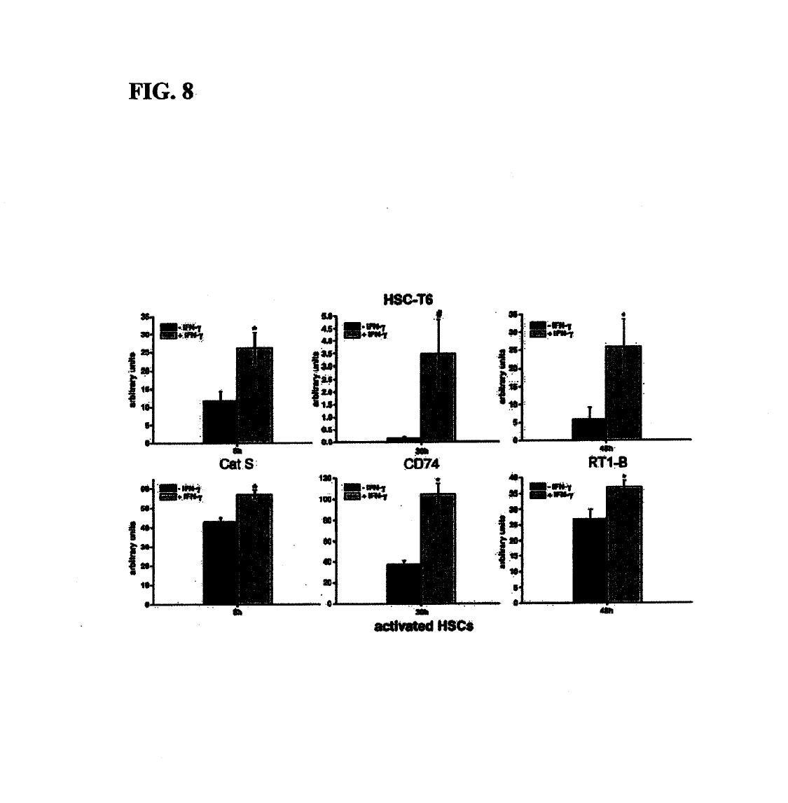

We have shown earlier on that there was an increase in the cathepsin S mRNA in HSC-T6 and activated HSCs treated with IFN-γ (FIG. 3A, 3B). At first it seems that our immunofluorescent approach failed to convincingly document an induction in cathepsin S at the antigen level at 8 h respectively for HSC-T6 and activated HSCs (FIG. 7A, 7C). FIG. 7 depicts the expression of cathepsin S in the IFN-γ treated and untreated HSC-T6 and activated HSCs. HSC-T6 and activated HSCs were plated and grown to a confluence of 60-70% overnight at conditions described. The cells were treated with IFN-γ for 8 h and stained by immunofluorescence as described. FIG. 7A and FIG. 7C represent the immunofluorescence with anti-cathepsin S antibody after 8 h of incubation with IFN-γ for HSC-T6 and activated HSCs respectively. FIG. 7B and FIG. 7D are the corresponding controls for the HSC-T6 and activated HSCs. Cells were counterstained with DAPI (blue). The scale bars in FIG. 7 are 10 μm. However, quantification of the fluorescence intensities from different areas of the same experiment showed that IFN-γ resulted also in a significant increase in cathepsin S expression on the protein level for HSC-T6 and the activated HSCs (FIG. 8). In FIG. 8, several fields of vision were used to quantify the total fluorescence with a boundary to the nuclei. The values were normalized to the area. Data are presented as mean±SD*P<0.05, #P<0.1.

Cathepsin S Activity Upon Induction with IFN-γ

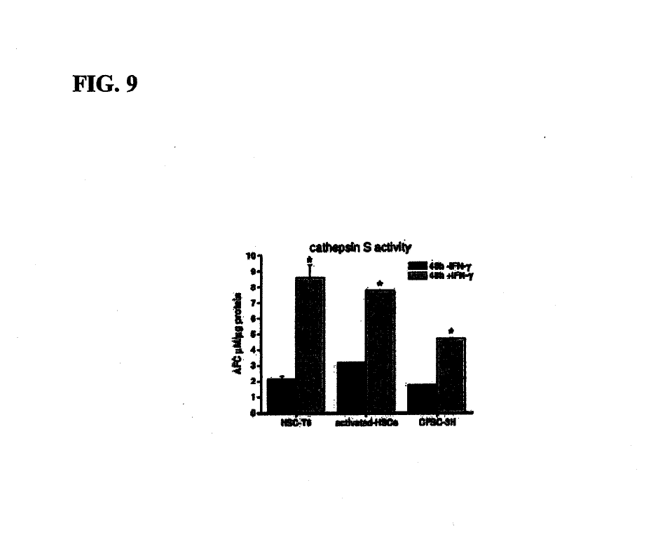

To further investigate the induction of cathepsin S by IFN-γ, activity measurement was performed to directly detect cathepsin S in activated HSCs and both cell lines. FIG. 9 depicts the cathepsin S activity in activated HSCs and two different cell lines. The cells were grown in cell culture medium containing 10% FBS. When they reached 50-60% confluence, IFN-γ was added or omitted as control. The cells were harvested 48 h later and resuspended in CS cell lysis buffer. Cathepsin S activity was measured using a final substrate concentration of 200 μM. The specific activity is given in released fluorophore (AFC) in μM per μg protein per hour at 37° C. Data are presented as mean±SD*P<0.05. There was a significant increase of cathepsin S specific activity in HSC-T6, activated HSCs and CFSC-3H (P<0.05) after 48 h (FIG. 9). This activity was almost completely inhibited by the cathepsin S inhibitor provided with the Biovision kit (data not shown).

Uptake and Processing of Ovalbumin

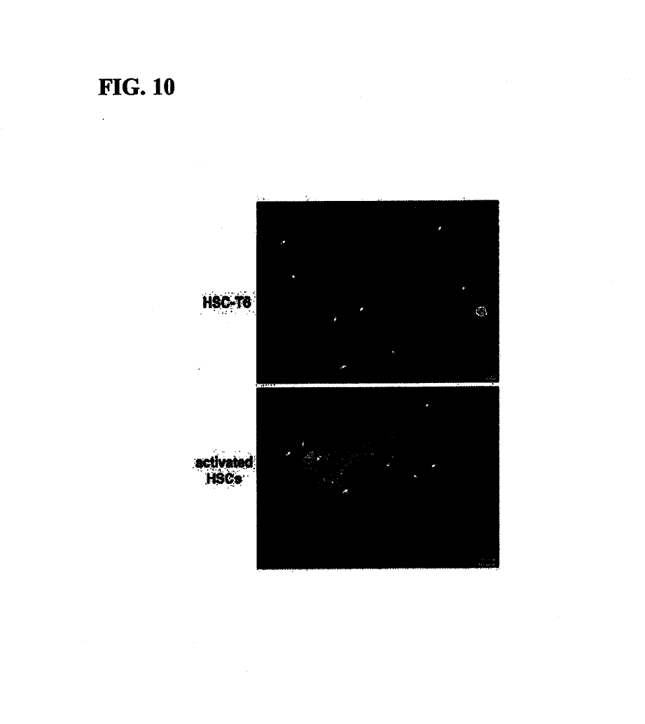

Successful antigen presentation requires the HSCs to internalize antigenic proteins and to process them into smaller peptides. To demonstrate these capabilities of activated HSCs and HSC-T6, a quenched tracer DQ-ovalbumin was used in the experiments. FIG. 10 depicts the uptake and processing of labeled ovalbumin. The cells were initially incubated with DQ-ovalbumin for 15 min at 37° C. and then washed twice with medium. Subsequently, the cells were incubated in medium alone for an additional 30 min, and imaged with a Leica epifluorescence microscope. The images show the uptake and digest of the DQ-ovalbumin by HSC-T6 and activated HSCs respectively. Arrows refer to the red-shifted excimer formed by high concentrations of digested ovalbumin. The scale bars are =10 μm.

DQ-ovalbumin is strongly labeled with the fluorescent BODIPY FL dye, whereby the fluorescence is quenched in the intact ovalbumin protein. Upon digestion into peptides, the fluorescence is released and can be detected with a standard fluorescein optical filter. The uptake of ovalbumin was thought to occur through receptor-mediated endocytosis by the Mannose receptor (Kindberg et al., 1990; Mousavi et al., 2005). In our case, the HSCs (activated HSCs as well as HSC-T6) took up the ovalbumin and processed it within 15 min (data not shown). After an additional processing time of 30 min, a shift in the fluorescence emission from green to orange became apparent as shown in FIG. 10. This shift was due to the formation of so-called excimer at spots with highly localized and concentrated digested peptide tracer, as described in the manufacturer's instructions. The activated HSCs showed a very strong green-yellow autofluorescence around the nucleus.

As a result, the green dots were not detectable, but orange stained vesicles became discernable after 30 min of incubation. This shows not only the uptake but also the successful processing of the antigen.

### Discussion

Recently published papers demonstrated by flow cytometric analysis that the MHC class II molecule (HLA-DR), and costimulatory molecules (such as CD40, CD80 and CD86) can be stimulated by IFN-γ in HSCs (Viñas et al. 2003; Yu et al 2004). However, no information was available concerning the early events of antigen presentation and about the molecules involved in these events. Among them, CIITA type IV the transactivator of class II molecules is considered as a ‘major regulator’ for other molecules, like MHC class II molecules and invariant chain (CD74), and is responsive to IFN-γ (LeibundGut-Landmann et al, 2004; Harton & Ting, 2000). Furthermore, the invariant chain was known to be involved in the assembly of the MHC class II molecules (Villadangos, 2001). As the invariant chain is blocking the antigen-binding pocket of the class II molecule, it has to be degraded by proteases. Two of the proteases involved in the processing are cathepsin L and cathepsin S, which participate, depending on the cell type (Nakagawa et al., 1998; Riese et al., 1998; Beers et al., 2005; Driessen et al., 1999), in the latter steps of degradation of the invariant chain.

The aim of the current study was to obtain more detailed information on the molecular mechanisms underlying antigen presentation in HSCs. Here, quantitative RT-PCR and immunofluorescence methods were employed to study the molecules involved in the early stage of antigen presentation.

Along with culture activated HSCs, we used in our study two cell lines. The HSC-T6 is a SV40 immortalized HSC cell line, regarded as semi-activated, whereas the CFSC-3H is derived from a cirrhotic liver and is regarded as in vivo activated. For the first time, we showed that activated HSCs, as well as the HSC cell lines HSC-T6 and CFSC-3H expressed transcripts for all molecules studied, namely CIITA, RT1-Bα, RT1-Dα, CD74, and cathepsin S (FIG. 2A-C). Interestingly we found that in addition to CIITA type IV (common to nonprofessional APCs), the CIITA type III was also expressed in HSCs. The transcript for CIITA type III was clearly detectable in activated HSCs and the HSC-T6 cell line, but not in CFSC-3H (FIG. 2A-C). This finding was particularly interesting as CIITA type III has been reported in another publication (Xu et al., 2004) to be induced by IFN-γ and subsequently mediated the repression of collagen (col1a2) in fibroblasts. We also discovered in the current study that the type III transcript in the HSCs was inducible by IFN-γ (data not shown). There could be some relationship between the expression of CIITA type III and the regulation of collagen expression in hepatic stellate cells. For the CFSC-3H we found solely the expression from the CIITA promoter type I (FIG. 2D). This type of CIITA is expressed in dendritic cells and IFN-γ induced macrophages (LeibundGut-Landmann et al, 2004). Whether this switch between the different CIITA promoters is a representation of the collagen production in fibrotic HSCs has to be studied. We did not pursue the question of the different expression of CIITA in this study, although this finding has potential in the perspective of the treatment of fibrotic HSCs. Apparently CIITA, being the master regulator for the MHCxclass II expression, was responding as expected. The increase inxmRNA level started early after addition of IFN-γ (FIG. 3A-C). The CIITA mediates the IFN-γ effect as shown by Steimle et al., (2004) and induced the increase in the mRNA levels of the MHC class II molecules and the invariant chain (CD74) as visualized at the later time-points (FIG. 3A-C). The immunofluorescence data for the CD74 and RT1-B molecules (FIG. 5 and FIG. 6) were in accordance with the quantitative RT-PCR data. The invariant chain was detectable after 24 h, but the difference in its expression between treated sample and control was best seen after 30 h (FIG. 5A, C). RT1-B expression however, was best detectable after 48 h (FIG. 6A, C). We quantified the fluorescence images and found a significant difference in the fluorescence intensities when comparing IFN-γ treated and untreated samples (FIG. 8).

The best studied function of cathepsin S is the processing of the invariant chain by releasing the CLIP from lip10 (Driessen et al., 1999). Therefore the finding that cathepsin S is expressed in HSCs and can be upregulated with the proinflammatory cytokine IFN-γ (FIG. 3A-C and FIG. 7) seems to suggest a possible contribution to the CD74 processing. This is substantiated by the increase in cathepsin S activity compared to the control (FIG. 9). On the other hand, cathepsin L which is another possible candidate for the final processing of the invariant chain (Nakagawa et al., 1998), showed no significant increase on the transcription level (FIG. 4). These results present the first indication towards a role of cathepsin S in HSCs.

While we investigated the response of CIITA, CD74, RT1-Dα and cathepsin S to IFN-γ using quantitative real-time PCR, we made another remarkable finding. Although the expression of these molecules increased after induction with IFN-γ, there were differences in the degree of their upregulation for the different HSCs (FIG. 3A-C). Without being limited to any particular theory, this phenomenon could be explained by the different basal expression of these molecules. Without being limited to any particular theory, the differences in origin of these cells, (cell culture activated HSCs, SV40 immortalization HSC-T6 and derivation from cirrhotic liver CFSC-3H) could also have an influence on the expression level. Without being limited to any particular theory, the stability of the transcripts could be differentially regulated in the various HSCs studied. Finally we showed that the hepatic stellate cells were capable of taking up antigenic proteins such as ovalbumin. More importantly, HSCs also own the molecular machinery needed to process them into smaller peptides (FIG. 10). The efficiency of this process was comparable in HSC-T6 and activated HSCs.

In conclusion, we have shown that activated hepatic stellate cells feature all molecules necessary for the early stage of antigen presentation. Furthermore, the HSCs are able to upregulate these molecules in response to IFN-γ, independent of their origin of activation. There was however a difference in the degree of upregulation. Another significant finding is that cathepsin S, a lysosomal cysteine protease primarily involved in the processing of CD74, was found in HSCs. This is important because this enzyme is a main target in treating autoimmune diseases (Yang et al., 2005) and seems to be involved in angiogenic processes (Shi et al, 2003; Wang et al., 2006). It is clear from this study that the lysosomal protein degradation is not the only function of cathepsin S. The finding that different CIITA promoters are used in HSC-T6, activated HSCs and the fibrotic CFSC-3H could point to another therapeutic target specific for fibrotic HSCs. We have also compared the HSC-T6 cell line with culture activated HSCs and concluded that this cell line retained many of the key features of the activated cells regarding antigen presentation. This cell line is thus suited for studying the molecular events that occurred during antigen presentation in HSCs.

### REFERENCES

M. Sato, S. Suzuki, H. Senoo, Hepatic stellate cells: unique characteristics in cell biology and phenotype, Cell Struct. Funct. 28 (2003) 105-112.
R. Bataller, D. A. Brenner, Hepatic stellate cells as a target for the treatment of liver fibrosis, Semin. Liver Dis. 21 (2001) 437-451.
S. Lotersztajn, B. Julien, F. Teixeira-Clerc, P. Grenard, A. Mallat, Hepatic fibrosis: molecular mechanisms and drug targets, Annu. Rev. Pharmacol. Toxicol. 45 (2005) 605-628.
O. Viñas, R. Bataller, P. Sancho-Bru, P. Ginès, C. Berenguer, C. Enrich, J. M. Nicolás, G. Ercilla, T. Gallart, J. Vives, V. Arroyo, J. Rodes, Human hepatic stellate cells show features of antigen-presenting cells and stimulate lymphocyte proliferation, Hepatology 38 (2003) 919-929.
M. C. Yu, C. H. Chen, X. Liang, L. Wang, C. R. Gandhi, J. J. Fung, L. Lu, S. Qian, Inhibition of T-cell responses by hepatic stellate cells via B7-H1-mediated T-cell apoptosis in mice, Hepatology 40 (2004) 1312-1321.
C. Gabay, I. Kushner, Acute-phase proteins and other systemic responses to inflammation, N. Engl. J. Med. 340 (1999) 448-454.
S. J. Wigmore, K. C. Fearon, J. P. Maingay, P. B. Lai, J. A. Ross, Interleukin-8 can mediate acute-phase protein production by isolated human hepatocytes, Am. J. Physiol. 273 (1997) E720-E726.
A. Limmer, J. Ohl, C. Kurts, H. G. Ljunggren, Y. Reiss, M. Groettrup, F. Momburg, B. Arnold, P. A. Knolle, Efficient presentation of exogenous antigen by liver endothelial cells to CD8+ Tcells results in antigen-specific T-cell tolerance, Nat. Med. 6 (2000) 1348-1354.
Y. Shiratori, K. Okano, K. Matsumoto, S. Murao, Antigen presentation by Kupffer cells in the rat, Scand. J. Gastroenterol. 19 (1984) 733-739.
C. R. Roland, L. Walp, R. M. Stack, M. W. Flye, Outcome of Kupffer cell antigen presentation to a cloned murine Th1 lymphocyte depends on the inducibility of nitric oxide synthase by IFN-gamma, J. Immunol. 153 (1994) 5453-5464.
P. J. O'Connell, A. B. Morelli, A. J. Logar, A. W. Thomson, Phenotypic and functional characterization of mouse hepatic CD8 alpha+ lymphoid-related dendritic cells, J. Immunol. 165 (2000) 795-803.
C. Johansson, M. J. Wick, Liver dendritic cells present bacterial antigens and produce cytokines upon Salmonella encounter, J. Immunol. 172 (2004) 2496-2503
A. H. Lau, A. W. Thomson, Dendritic cells and immune regulation in the liver, Gut 52 (2003) 307-314.
S. LeibundGut-Landmann, J. M. Waldburger, M. Krawczyk, L. A. Otten, T. Suter, A. Fontana, H. Acha-Orbea, W. Reith, Mini-review: specificity and expression of CIITA, the master regulator of MHC class II genes, Eur. J. Immunol. 34 (2004) 1513-1525
T. Nakagawa, W. Roth, P. Wong, A. Nelson, A. Farr, J. Deussing, J. A. Villadangos, H. Ploegh, C. Peters, A. Y. Rudensky, Cathepsin L: critical role in Ii degradation and CD4 T cell selection in the thymus, Science 280 (1998) 450-453.
R. J. Riese, R. N. Mitchell, J. A. Villadangos, G. P. Shi, J. T. Palmer, E. R. Karp, G. T. De Sanctis, H. L. Ploegh, H. A. Chapman, Cathepsin S activity regulates antigen presentation and immunity, J. Clin. Invest. 101 (1998) 2351-2363.
C. Beers, A. Burich, M. J. Kleijmeer, J. M. Griffith, P. Wong, A. Y. Rudensky, Cathepsin S controls MHC class II-mediated antigen presentation by epithelial cells in vivo, J. Immunol. 174 (2005) 1205-1212.
C. Driessen, R. A. Bryant, A. M. Lennon-Dumenil, J. A. Villadangos, P. W. Bryant, G. P. Shi, H. A. Chapman, H. L. Ploegh, Cathepsin S controls the trafficking and maturation of MHC class II molecules in dendritic cells, J. Cell Biol. 147 (1999) 775-790.
L. Riccalton-Banks, R. Bhandari, J. Fry, K. M. Shakesheff, A simple method for the simultaneous isolation of stellate cells and hepatocytes from rat liver tissue, Mol. Cell. Biochem. 248 (2003) 97-102.
S. Vogel, R. Piantedosi, J. Frank, A. Lalazar, D. C. Rockey, S. L. Friedman, W. S. Blaner, An immortalized rat liver stellate cell line (HSC-T6): a new cell model for the study of retinoid metabolism in vitro, J. Lipid Res. 41 (2000) 882-893.
V. Steimle, C. A. Siegrist, A. Mottet, B. Lisowska-Grospierre, B. Mach, Regulation of MHC class II expression by interferon-gamma mediated by the transactivator gene CIITA, Science 265 (1994) 106-109.
A. M. Lennon, C. Ottone, G. Rigaud, L. L. Deaven, J. Longmire, M. Fellous, R. Bono, C. Alcaide-Loridan, Isolation of a B-cell-specific promoter for the human class II transactivator, Immunogenetics 45 (1997) 266-273.
T. Flannery, D. Gibson, M. Mirakhur, S. McQuaid, C. Greenan, A. Trimble, B. Walker, D. McCormick, P. G. Johnston, The clinical significance of cathepsin S expression in human astrocytomas, Am. J. Pathol. 163 (2003) 175-182.
J. M. Soos, J. I. Krieger, O. Stuve, C. L. King, J. C. Patarroyo, K. Aldape, K. Wosik, A. J. Slavin, P. A. Nelson, J. P. Antel, S. S. Zamvil, Malignant glioma cells use MHC class II transactivator (CIITA) promoters III and IV to direct IFN-gamma-inducible CIITA expression and can function as nonprofessional antigen presenting cells in endocytic processing and CD4(+) T-cell activation, Glia 36 (2001) 391-405.
K. Storm van's Gravesande, M. D. Layne, Q. Ye, L. Le, R. M. Baron, M. A. Perrella, L. Santambrogio, E. S. Silverman, R. J. Riese, IFN regulatory factor-1 regulates IFN-gamma-dependent cathepsin S expression, J. Immunol. 168 (2002) 4488-4494.
M. A. Rahat, I. Chemichovski, N. Lahat, Increased binding of IFN regulating factor 1 mediates the synergistic induction of CIITA by IFN-γ and tumor necrosis factor-α in human thyroid carcinoma cells, Int. Immunol. 13 (2001) 1423-1432.
J. A. Harton, J. P. Ting, Class II transactivator: mastering the art of major histocompatibility complex expression, Mol. Cell. Biol. 20 (2000) 6185-6194.
G. M. Kindberg, S. Magnusson, T. Berg, B. Smedsrod, Receptor-mediated endocytosis of ovalbumin by two carbohydrate-specific receptors in rat liver cells. The intracellular transport of ovalbumin to lysosomes is faster in liver endothelial cells than in parenchymal cells, Biochem. J. 270 (1990) 197-203.
S. A. Mousavi, M. Sato, M. Sporstol, B. Smedsrod, T. Berg, N. Kojima, H. Senoo, Uptake of denatured collagen into hepatic stellate cells: evidence for the involvement of urokinase plasminogen activator receptorassociated protein/Endo180, Biochem. J. 387 (2005) 39-46.
J. A. Villadangos, Presentation of antigens by MHC class II molecules: getting the most out of them, Mol. Immunol. 38 (2001) 329-346.
Y. Xu, L. Wang, G. Buttice, P. K. Sengupta, B. D. Smith, Major histocompatibility class II transactivator (CIITA) mediates repression of collagen (COL1A2) transcription by interferon gamma (IFN-gamma), J. Biol. Chem. 279 (2004) 41319-41332.
H. Yang, M. Kala, B. G. Scott, E. Goluszko, H. A. Chapman, P. Christadoss, Cathepsin S is required for murine autoimmune myasthenia gravis pathogenesis, J. Immunol. 174 (2005) 1729-1737.
G. P. Shi, G. K. Sukhova, M. Kuzuya, Q. Ye, J. Du, Y. Zhang, J. H. Pan, M. L. Lu, X. W. Cheng, A. Iguchi, S. Perrey, A. M. E. Lee, H. A. Chapman, P. Libby, Deficiency of the cysteine protease cathepsin S impairs microvessel growth, Circ. Res. 92 (2003) 493-500.
B. Wang, J. Sun, S. Kitamoto, M. Yang, A. Grubb, H. A. Chapman, R. Kalluri, G. P. Shi, Cathepsin S controls angiogenesis and tumor growth via matrix-derived angiogenic factors, J. Biol. Chem. 281 (2006) 6020-6029.
S. L. Friedman Liver fibrosis—from bench to bedside, J. Hepatol 38 Suppl 1 (2003):S38-S53.
D. Cassiman, L. Libbrecht., V. Desmet, C. Denef, T. Roskams, Hepatic stellate cell/myofibroblast subpopulations in fibrotic human and rat livers. J. Hepatol. 36(2) (2002) 200-9.
A. Geerts, On the origin of stellate cells: mesodermal, endodermal or neuro-ectodermal? J. Hepatol. 40(2) (2004) 31-4.
R. Bataller, D. A. Brenner, Liver fibrosis, J. Clin. Invest. 115(2) (2005),
S. L. Friedman, D C Rockey, R F McGuire, J J Maher, J K Boyles, G Yamasaki. Isolated hepatic lipocytes and Kupffer cells from normal human liver: morphological and functional characteristics in primary culture Hepatology 15(2) (1992) 234-43.
D. Cassiman, J. van Pelt, R. De Vos, F. van Lommel, V. Desmet, S. H. Yap, T. Roskams, Synaptophysin: A novel marker for human and rat hepatic stellate cells AM. J. Pathol. 155(6) (1999) 1831-9.
L. Xu, A. Y. Hui, M. J. Albanis, S. M. O'Byrne, W. S. Blaner, P. Mukherjee, S. L. Friedman, F. J. Eng, Human hepatic stellate cell lines, LX-1 and LX-2: new tools for analysis of hepatic fibrosis. Gut 54(1) (2005) 142-51.
Burt A. D., Pathobiology of hepatic stellate cells, J. Gasterenterol 34(3) (1999) 299-304.
R. L. Thurmond, S. Sun, C. A. Sehon, S. M. Baker, H. Cai, Y. Gu, W. Jiang, J. P. Riley, K. N. Williams, J. P. Edwards, and L. Karlsson, Identification of a Potent and Selective Noncovalent Cathepsin S Inhibitor, Journal of Pharmacology And Experimental Therapeutics 308 (1) (2004) 268-276.
N. Katanuma, E. Murata, H. Kakegawa, A. Matsui, H. Tsuzuki, H. Tsuge, D. Turk, V. Turk, M. Fukushima, Y. Tada, T. Asao, Structure based development of novel specific inhibitors for cathepsin L and cathepsin S in vitro and in FEBS Letters 458 (1999) 6-10.
F. Winau, G. Hegasy, R. Weiskirchen, S. Weber, C. Cassan, P. A. Sieling, R. L. Modlin, R. S. Liblau, A. M. Gressner and S. H. E. Kaufmann, Ito Cells Are Liver-Resident Antigen-Presenting Cells for Activating T Cell Responses Immunity 26 (2007) 117-129.

1. A method of identifying a hepatic anti-fibrotic agent or hepatic anti-inflammatory agent, the method comprising:
a) determining a first expression level of Cat S in a first activated hepatic stellate cell;
b) exposing a second activated hepatic stellate cell to a test compound;
c) determining a second expression level of Cat S in said second activated hepatic stellate cell;
d) comparing the first expression level and the second expression level
whereby the first expression level which is greater than the second expression level indicates that the test compound is a hepatic anti-fibrotic agent or hepatic anti-inflammatory agent.

2. The method of claim 1 further comprising the step of exposing the first and second hepatic stellate cells to a cytokine prior to determining the first and second expression levels.

3. The method of claim 1 wherein the first hepatic stellate cell and the second hepatic stellate cell are provided in vitro.

4. The method of claim 3 wherein the first hepatic stellate cell and the second hepatic stellate cell are a HSC-T6 cell.

5. The method of claim 2 wherein the cytokine is IFN-γ.

6. A kit comprising:
a) a hepatic stellate cell; and
b) a reagent for detecting the expression level of Cat S.

7. The kit according to claim 6 wherein the reagent for detecting the expression level of Cat S is a nucleic acid complementary to a portion of the Cat S gene.

8. The kit according to claim 6 wherein the reagent for detecting the expression level of Cat S is an anti-cathepsin S antibody.

9. The kit according to claim 8 wherein the antibody is a monoclonal antibody.

10. The kit according to claim 6 wherein the hepatic stellate cell is a HSC-T6 cell.

11. The kit according to claim 6 further comprising a cytokine.

12. The kit according to claim 11 wherein the cytokine is IFN-γ.
